(19) United States
(12) Patent Application Publication
(10) Pub. No.: US 2009/0062620 A1
(43) Pub. Date: Mar. 5, 2009

(54) Methods for Estimating the Metabolic Cost of Walking

(75) Inventors: Peter G. Weyand, Houston, TX (US); Bethany R. Smith, Houston, TX (US); Nancy Butte, Houston, TX (US)

Correspondence Address: Tod T. Tumey, P.O. BOX 22188, HOUSTON, TX, 77227-2188, US

(73) Assignees: WILLIAM MARSH RICE UNIVERSITY, Houston, TX (US); Baylor College of Medicine, Houston, TX (US)

(21) Appl. No.: 11/848,085

(22) Filed: Aug. 30, 2007

(51) Int. Cl. A61B 5/00

(52) U.S. Cl. 600/300; 128/898

(57) ABSTRACT

Methods for determining the metabolic cost for walking are disclosed. In one embodiment, a method for determining metabolic cost per unit time of a walk by an individual is disclosed. The method includes providing a body length of the individual. The method also includes measuring a walking speed of the individual. The metabolic cost per unit time is determined by adding a basal metabolic rate and a metabolic constant for walking to the result of a metabolic coefficient for walking multiplied by the walking speed (squared) and such squared speed divided by the body length of the individual.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

This invention relates to the field of exercise and more specifically to the metabolic cost of walking.

2. Background of the Invention

Physical fitness is in increasingly high demand. Such a demand has also increased interest in walking for physical fitness. Moreover, participants in walking for exercise have been interested in determining the amount of energy expended during the walking exercise.

Techniques have been developed to determine the amount of energy expended during a walking exercise. For instance, conventional techniques have been developed to determine the amount of calories burned during the exercise. Drawbacks to such conventional techniques include such techniques typically using only body weight and speed to estimate the calories expended. Such conventional techniques typically do not take into account body size such as height and therefore may be inaccurate.

Consequently, there is a need for an improved method for determining the metabolic cost of walking. Further needs include an improved method for determining metabolic cost that takes into account body size.

### BRIEF SUMMARY OF SOME OF THE PREFERRED EMBODIMENTS

These and other needs in the art are addressed in one embodiment by a method for determining metabolic cost per unit time of a walk by an individual. The method includes providing a body length of the individual. The method also includes measuring a walking speed of the individual. The metabolic cost per unit time is determined by the following equation:

BMR+MBC+MBCo*(spd²/BL).

In the equation, BMR is basal metabolic rate, MBC is a metabolic constant for walking, MBCo is a metabolic coefficient for walking, speed is the walking speed, and BL is the body length.

In other embodiments, these and other needs in the art are addressed by a method for determining metabolic cost of a walk by an individual. The method includes providing a body length of the individual. The method also includes measuring weight of the individual. The method further includes measuring a distance traveled during the walk. The metabolic cost is determined by the following equation:

WC*BLT*Wt.

In the equation, WC is a walking constant, BLT is number of body lengths traveled during the walk, and Wt is the weight of the individual.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates representative symbols for Groups A-E as used in FIGS. 2-4;

FIG. 2 illustrates the metabolic cost per speed for Groups A-E;

FIG. 3 illustrates the metabolic cost of transport per speed for Groups A-E; and

FIG. 4 illustrates the product of the metabolic cost of transport by height per speed for Groups A-E.

### DETAILED DESCRIPTION OF THE PREFERRED EMBODIMENTS

It has been discovered that taking into account the body length of a walking individual provides an improved method of determining the metabolic cost of walking. Metabolic cost refers to the metabolic energy expended during a walking exercise. In an embodiment, body length refers to the height of the walking individual. It is to be understood that height is measured with the walking individual in a standing and upright position. In other embodiments, body length refers to the leg length of the walking individual. It is to be understood that leg length refers to the length of a leg of the walking individual as measured from the base of the foot of the measured leg to the axis of rotation of the hip joint. It is to be further understood that walking refers to locomotion of the individual over a surface by movement of the legs of the individual on the surface and may not include an aerial phase (e.g., with walking, at least one foot is in contact with the surface). In an embodiment, walking comprises a speed of about 3.5 miles/hour or less, alternatively a speed of about 4.5 miles/hour or less. Without being limited by theory, the metabolic cost of walking is directly dependent on body length. For instance, without being limited by theory, it is believed that the rate at which each gram of body tissue metabolizes chemical energy during walking may be directly related to the body length (e.g., height).

In an embodiment, the metabolic cost per unit of time of walking by a walking individual may be determined by adding the basal metabolic rate and the metabolic constant for walking to the result of the metabolic coefficient for walking multiplied by the result of the speed of the walking individual (squared) divided by the body length (e.g., leg length or height) of the walking individual. For instance, the metabolic cost of a walk per unit of time may be determined by the following Equation (1).

(E/t)=BMR+MBC+MBCo*(spd²/BL)

In Equation (1), E is the metabolic cost, t is time, BMR is the basal metabolic rate, MBC is metabolic constant for walking, MBCo is metabolic coefficient for walking, spd is the walking speed of the walking individual, and BL is the body length of the walking individual. BMR refers to the rate at which energy is used by an individual at complete rest and in a post-absorptive state. BMR may be determined using any suitable method. It is to be understood that different information about the walking individual may be used for different methods. For instance, suitable methods for determining BMR may use the weight of the walking individual; the weight and height of the walking individual; the weight, height, and gender of the walking individual; or the weight, height, gender, and age of the walking individual. In an embodiment, BMR is determined using the weight and height of the walking individual; the weight, height, and gender of the walking individual; or the weight, height, gender, and age of the walking individual. In some embodiments, BMR is determined using the weight, height, and gender of the walking individual or the weight, height, gender, and age of the walking individual. In other embodiments, BMR is determined using the weight, height, gender, and age of the walking individual. BMR may be determined using any suitable method including methods using such different information about the walking individual. For instance, without limitation, BMR may be determined using gas exchange or direct heat measurements. Without limitation, examples of suitable methods for determining BMR include the methods disclosed in Schofield, W N, Schofield E. and James W P T, Predicting Basal Metabolic Rate: New Standards and a Review of Previous Work, Human Nutrition, Clinical Nutrition, 39, Supplement 1, pgs. 5-41 (1985), which is incorporated by reference herein in its entirety. This article is referred to as “Schofield” for purposes of this application. MBC is the minimum energy required to be expended by an individual to maintain a walking posture (e.g., standing upright without locomotion across the surface). In an embodiment, MBC is 3.27 mls of oxygen/(kg of body mass*minute), alternatively 65.7 J/(kg of body mass*minute). In an embodiment, BL is the height of the walking individual. In such an embodiment in which BL is the height of the walking individual, MB Co is 5.63 mls of oxygen/(kg of body mass*minute), alternatively 113.2 J/(kg of body mass*minute). In other embodiments, BL is the leg length of a leg of the walking individual. In such other embodiments in which BL is the leg length of a leg of the walking individual, MBCo is 2.96 mls of oxygen/(kg of body mass*minute), alternatively 59.7 J/(kg of body mass*minute). It is to be understood that BMR may differ with each walking individual, but MBC and MBCo may not differ from each walking individual. It has been found that Equation (1) may determine E/t of the walking individual with about a ±5% error to the actual E/t. It is to be understood that all components of Equation (1) may be converted to similar units for obtaining the results of Equation (1).

In regards to Equation (1), the total metabolic cost for a walk may be determined. For instance, to determine the total metabolic cost during the duration of a walk by the walking individual (e.g., total energy expended), all of the determined E/t results from Equation (1) from the duration of the walk (e.g., total time of the walk) may be summed together. In alternative embodiments, the total metabolic cost of the walk may be determined by multiplying E/t by the duration of the walk. In some embodiments, the total metabolic cost of a walk may be estimated for a determined E/t by multiplying such determined E/t by the expected duration of the walk. For instance, an individual may be walking, and the E/t for a desired unit of time is determined using Equation (1). The estimated total metabolic cost for a desired walking duration may then be determined by multiplying such desired walking duration by the determined E/t.

In another embodiment, the metabolic cost for a walk is determined by a method that includes the weight of the walking individual, the body length of the walking individual, and the distance traveled by the walking individual. In such an embodiment, the metabolic cost is determined by multiplying the number of body lengths traveled by a walking constant and the weight of the walking individual as shown by the following Equation (2).

E=WC*BLT*Wt

In regards to Equation (2), E is the metabolic cost, WC is a walking constant, BLT is the number of body lengths traveled during the walk, and Wt is the weight of the walking individual. WC is 0.97 cal/kg. It is to be understood that WC may be converted into similar units as the other components of Equation (2). BLT is determined by dividing the distance traveled by the body length of the walking individual. In an embodiment, the body length is the height of the walking individual. In other embodiments, the body length is the combined length of both of the legs of the walking individual, alternatively the body length is the length of one of the legs of the walking individual multiplied by 2. It is to be understood that all components of Equation (2) may be converted to similar units for obtaining the results of Equation (2).

It is to be understood that any method in which the walking individual is walking may be used with Equations (1), (2), or both to determine the metabolic cost. Without limitation, the walking individual may be walking on a treadmill, using a pedometer, using a Global Positioning System (GPS), and/or a program (e.g., software program). For instance, in regards to a treadmill, the treadmill may be programmed with sufficient programming to determine the metabolic cost using Equations (1) and/or (2). The treadmill may also be programmed to receive input regarding information about the walking individual used for the Equations (e.g., weight, body length, and the like). The treadmill may then measure and record information about the walk such as duration, speed, distance, and the like, which may be used in Equations (1) and/or (2) along with the input information to determine the metabolic cost. In some embodiments, the walking individual may measure sufficient information for Equations (1) and/or (2) and then input such measured information in a software program to determine the metabolic cost of the walk. It is to be understood that the software program is sufficiently programmed to determine E and/or E/t using Equations (1) and/or (2). For instance, the walking individual may measure information such as the distance traveled, speed of the walk, and duration of the walk. Such information may be measured by any suitable method such as GPS, pedometer, and the like. The walking individual may also measure the weight and body length (e.g., height) of the walking individual. All of such measured information may be input into a software program to determine the metabolic cost. In some embodiments, the software program may be integrated into the method of measuring the information regarding the walk. For instance, the software program may be integrated into the GPS or the pedometer. In other embodiments, the software program may be available on a compact disk or on a website, with sufficient information input into the program to provide the results using Equations (1) and/or (2).

To further illustrate various illustrative embodiments of the present invention, the following examples are provided.

### EXAMPLE

In this example, metabolic rates were measured at walking speeds from 0.4 to 1.9 m/s in human subjects that varied in height from 1.07 to 2.06 meters and varied in weight from 20.0 kg to 118.0 kg. The walking durations ranged from 4 to 6 minutes on a treadmill with oxygen uptake (e.g., indirect calorimetry) being measured continuously. Constants were determined from the data of the tested individuals. Each of Tables 1-5 shows the average data for all of the walking individuals in a particular group at a particular measured walking speed. The Groups included the following number of individuals: Group A (7 individuals); Group B (13 individuals); Group C (18 individuals); Group D (24 individuals); and Group E (7 individuals). Metabolic rates were determined from rates of oxygen uptake measured under steady-state conditions using indirect calorimetry and an energetic equivalent of 20.1 Joules per milliliter of oxygen. The subjects were divided into 5 groups (Groups A-E) on the basis of height. For Groups A-D, BMR was determined based on height, weight, gender, and age using the methods taught by Schofield. The BMR used for Group E was 3.5. Groups A-E are reflected in Tables I-V below.

TABLE I
Group A

| | | Height | | Leg | | | Standard | | Standard | | |
|---|---|---|---|---|---|---|---|---|---|---|---|
| Height | Age | Average | Weight | Length | Speed | VO₂/kg | Error | COT | Error | | Standard Error |
| (cm) | (yrs) | (m) | (kg) | (m) | (m/s) | (W/kg) | VO₂/kg | (J/kg/m) | COT | COT * Ht(J/kg) | COT * Ht |
| 107-122 | 5.43 | 1.14 | 21.27 | 0.58 | 0.4 | 4.18 | 0.25 | 5.17 | 0.36 | 5.88 | 0.38 |
| | | | | | 0.7 | 4.64 | 0.23 | 3.60 | 0.18 | 4.11 | 0.18 |
| | | | | | 1.0 | 5.28 | 0.23 | 3.17 | 0.14 | 3.61 | 0.12 |
| | | | | | 1.3 | 6.72 | 0.36 | 3.54 | 0.21 | 4.03 | 0.18 |

TABLE II
Group B

| | | Height | | Leg | | | Standard | | Standard | | |
|---|---|---|---|---|---|---|---|---|---|---|---|
| Height | Age | Average | Weight | Length | Speed | VO₂/kg | Error | COT | Error | | Standard Error |
| (cm) | (yrs) | (m) | (kg) | (m) | (m/s) | (W/kg) | VO₂/kg | (J/kg/m) | COT | COT * Ht(J/kg) | COT * Ht |
| 130-150 | 10.15 | 1.42 | 44.12 | 0.77 | 0.4 | 3.05 | 0.10 | 3.87 | 0.14 | 5.49 | 0.20 |
| | | | | | 0.7 | 3.52 | 0.10 | 2.88 | 0.10 | 4.08 | 0.12 |
| | | | | | 1.0 | 4.03 | 0.11 | 2.53 | 0.08 | 3.59 | 0.11 |
| | | | | | 1.3 | 4.99 | 0.13 | 2.68 | 0.09 | 3.80 | 0.11 |
| | | | | | 1.6 | 6.65 | 0.24 | 3.19 | 0.14 | 4.51 | 0.17 |

TABLE III
Group C

| | | Height | | Leg | | | Standard | | Standard | | |
|---|---|---|---|---|---|---|---|---|---|---|---|
| Height | Age | Average | Weight | Length | Speed | VO₂/kg | Error | COT | Error | | Standard Error |
| (cm) | (yrs) | (m) | (kg) | (m) | (m/s) | (W/kg) | VO₂/kg | (J/kg/m) | COT | COT * Ht(J/kg) | COT * Ht |
| 151-168 | 18.22 | 1.61 | 58.42 | 0.85 | 0.4 | 2.63 | 0.08 | 3.56 | 0.14 | 5.73 | 0.23 |
| | | | | | 0.7 | 3.07 | 0.08 | 2.68 | 0.09 | 4.30 | 0.15 |
| | | | | | 1.0 | 3.47 | 0.09 | 2.67 | 0.07 | 3.65 | 0.11 |
| | | | | | 1.3 | 4.19 | 0.11 | 2.30 | 0.07 | 3.70 | 0.11 |
| | | | | | 1.6 | 5.40 | 0.16 | 2.62 | 0.08 | 4.23 | 0.13 |
| | | | | | 1.9 | 6.65 | 0.28 | 2.87 | 0.13 | 4.70 | 0.22 |

TABLE IV
Group D

| | | Height | | Leg | | | Standard | | Standard | | |
|---|---|---|---|---|---|---|---|---|---|---|---|
| Height | Age | Average | Weight | Length | Speed | VO₂/kg | Error | COT | Error | | Standard Error |
| (cm) | (yrs) | (m) | (kg) | (m) | (m/s) | (W/kg) | VO₂/kg | (J/kg/m) | COT | COT * Ht(J/kg) | COT * Ht |
| 170-190 | 23.58 | 1.78 | 76.36 | 0.94 | 0.4 | 2.58 | 0.20 | 3.72 | 0.12 | 6.62 | 0.24 |
| | | | | | 0.7 | 2.93 | 0.06 | 2.64 | 0.11 | 4.70 | 0.20 |
| | | | | | 1 | 3.27 | 0.08 | 2.18 | 0.05 | 3.89 | 0.09 |
| | | | | | 1.3 | 3.90 | 0.14 | 2.17 | 0.06 | 3.85 | 0.12 |
| | | | | | 1.6 | 4.95 | 0.22 | 2.41 | 0.09 | 4.29 | 0.15 |
| | | | | | 1.9 | 6.35 | 0.16 | 2.77 | 0.08 | 4.92 | 0.13 |

TABLE V
Group E

| | | Height | | Leg | | | Standard | | Standard | | |
|---|---|---|---|---|---|---|---|---|---|---|---|
| Height | Age | Average | Weight | Length | Speed | VO₂/kg | Error | COT | Error | | Standard Error |
| (cm) | (yrs) | (m) | (kg) | (m) | (m/s) | (W/kg) | VO₂/kg | (J/kg/m) | COT | COT * Ht(J/kg) | COT * Ht |
| 199-206 | 21.71 | 2.04 | 99.34 | 1.12 | 0.4 | 2.37 | 0.10 | 3.00 | 0.25 | 6.12 | 0.53 |
| | | | | | 0.7 | 2.78 | 0.15 | 2.30 | 0.21 | 4.69 | 0.45 |
| | | | | | 1 | 3.07 | 0.15 | 1.89 | 0.15 | 3.85 | 0.32 |
| | | | | | 1.3 | 3.64 | 0.17 | 1.90 | 0.13 | 3.87 | 0.27 |
| | | | | | 1.6 | 4.54 | 0.21 | 2.11 | 0.13 | 4.29 | 0.28 |
| | | | | | 1.9 | 5.72 | 0.27 | 2.39 | 0.14 | 4.87 | 0.29 |

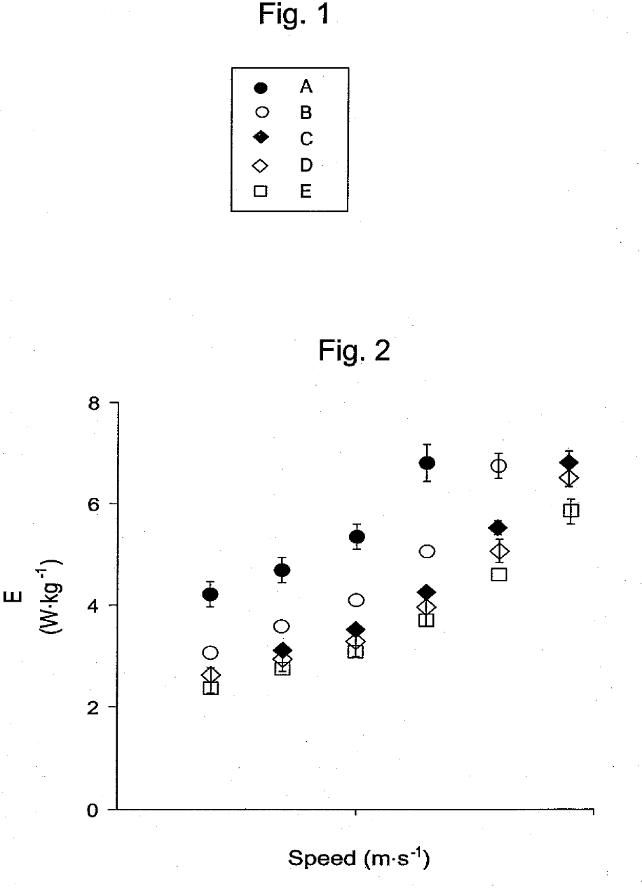

The gross metabolic rates of the subjects, expressed in relation to body mass per convention and analysis, conformed to expected patterns. As shown in Tables I-V, the rates increased in a curvilinear manner with increased walking sped. Consequently, the energy expended per unit distance (e.g., the metabolic rate/rate of forward speed) was minimized at intermediate walking speeds. On a mass-specific basis, shorter individuals expended more energy than taller individuals. FIG. 1 illustrates the representative symbols of Groups A-E as used in FIGS. 2-4. Metabolic rates increased in a curvilinear fashion with walking speed for all subjects and regardless of stature as shown in FIG. 2, with the metabolic rate about doubling from the slowest to the fastest walking speed within each group. Group means at each speed were directly related to height, with the means about twice for the shortest group (Group A) than the tallest group (Group E) at common speeds. The shortest subjects of Group A had the highest mass-specific metabolic rates, the tallest subjects of Group E had the lowest metabolic rates, and the rates of the three intermediate Groups B-D were inversely related to the height of the group.

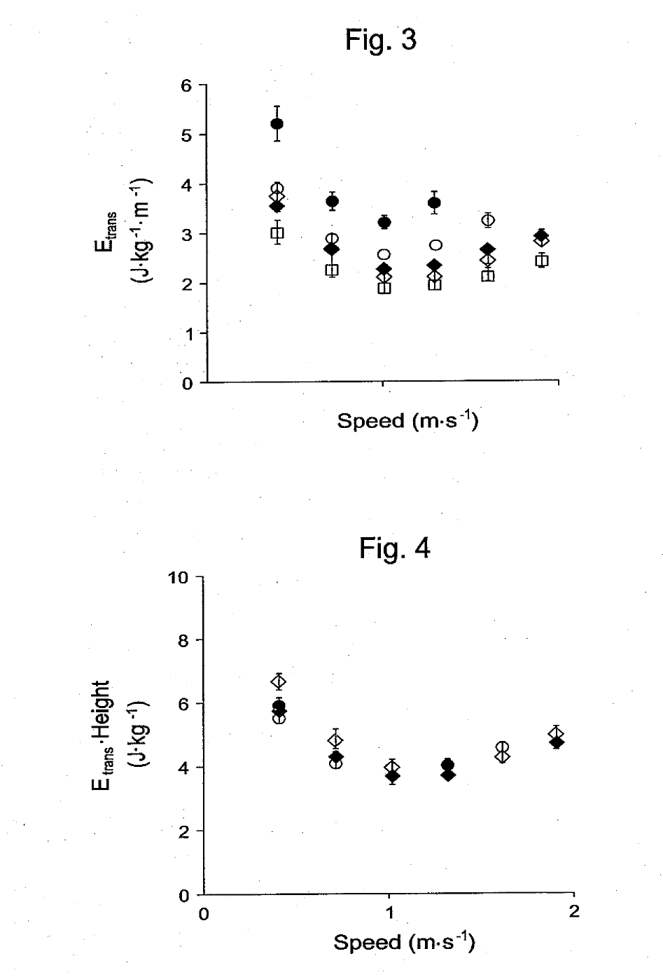

To isolate the proportion of the total metabolic signal incurred above that required by the body's non-locomotor tissues, the rates of metabolism needed to maintain the non-locomotor tissues under basal conditions were subtracted. The BMR was determined using methods of Schofield. Such subtraction was carried out in direct accordance with the disclosure of Schmidt-Neilsen, K., Locomotion: Energy Cost of Swimming, Flying, and Running, Science, pgs. 177 (45):222-8 (Jul. 21, 1972) to partition gross metabolic rates into resting and locomotor portions. This article is referred to as “Schmidt-Nielsen” for purposes of this application. The publication of Schmidt-Neilsen is incorporated herein by reference in its entirety. The locomotor metabolic rates obtained by speed were divided to determine the energy expended per unit distance traveled, or the metabolic cost of transport as taught by Schmidt-Neilsen and Taylor et al., Energetics and Mechanics of Terrestrial Locomotion, 1. Metabolic Energy Consumption as a Function of Speed and Body Size in Birds and Mammals, J. Exp. Biol., 97: 1-21 (April 1982). The publication of Taylor et al. is incorporated by reference herein in its entirety. Transport costs for all of the groups spanned roughly a 1.5-fold range and were minimized at the intermediate speeds at which humans typically choose to travel as shown in FIG. 3 and disclosed in Bornstein & Bornstein, The Pace of Life, Nature Publishing Group, Nature vol. 259, pgs. 557-559 (Feb. 19, 1976). Bornstein & Bornstein is incorporated by reference herein in its entirety. Transport costs followed the height dependency of gross metabolic rates about identically, spanning about a two-fold range between the shortest and tallest groups (Groups A and E, respectively). Group means at each speed and for 26 of the 28 total comparisons were inversely related to height.

To assess the influence of height on locomotor transport costs directly, the mass-specific transport costs per unit distance to the length or stature of the body were standardized. These dimensionless transport costs, representing the mass-specific locomotor cost of transporting one kilogram a horizontal distance equal to the body's length is shown in FIG. 4.

As shown in FIG. 2, metabolic rates increased in a curvilinear fashion. The non-linear relationship between E and speed lead to a characteristic shallow parabola shaped curve in the metabolic cost of transport for the speed as shown in FIG. 3. In addition, the product of the cost of transport and height as shown in FIG. 4 eliminated the difference in metabolic cost between height groups.

Although the present invention and its advantages have been described in detail, it should be understood that various changes, substitutions and alterations may be made herein without departing from the spirit and scope of the invention as defined by the appended claims.

1. A method for determining metabolic cost per unit time of a walk by an individual, comprising:
(A) providing a body length of the individual; and
(B) measuring a walking speed of the individual, wherein the metabolic cost per unit time is determined by:
BMR+MBC+MBCo*(spd²/BL),
wherein BMR is basal metabolic rate, MBC is a metabolic constant for walking, MBCo is a metabolic coefficient for walking, spd is the walking speed, and BL is the body length.

2. The method of claim 1, further comprising determining the BMR.

3. The method of claim 2, wherein the BMR is determined from information comprising weight of the individual, height of the individual, gender of the individual, and age of the individual.

4. The method of claim 1, wherein MBC is 65.7 J/(kg of body mass*minute).

5. The method of claim 1, wherein the body length is height of the individual.

6. The method of claim 5, wherein MBCo is 5.63 mls of oxygen/(kg of body mass*minute).

7. The method of claim 1, wherein the body length is leg length of a leg of the individual.

8. The method of claim 7, wherein MBCo is 2.96 mls of oxygen/(kg of body mass*minute).

9. The method of claim 1, further comprising determining total metabolic cost of the walk.

10. The method of claim 9, wherein the total metabolic cost of the walk is determined by summing together all of the determined metabolic cost per unit times for a duration of the walk.

11. The method of claim 9, wherein the total metabolic cost of the walk is determined by multiplying the determined metabolic cost per unit time by duration of the walk.

12. The method of claim 1, further comprising estimating a total metabolic cost of the walk, wherein the determined metabolic cost per unit time is multiplied by an estimated duration of the walk to provide an estimated total metabolic cost.

13. The method of claim 1, wherein the walk comprises a speed of about 4.5 miles/hour or less.

14. A method for determining metabolic cost of a walk by an individual, comprising:
(A) providing a body length of the individual;
(B) measuring a weight of the individual; and
(C) measuring a distance traveled during the walk, wherein the metabolic cost is determined by:
WC*BLT*Wt,
wherein WC is a walking constant, BLT is number of body lengths traveled during the walk, and Wt is the weight of the individual.

15. The method of claim 14, wherein BLT is determined by dividing the distance traveled by the body length of the individual.

16. The method of claim 14, wherein WC is 0.97 cal/kg.

17. The method of claim 14, wherein the body length is height of the individual.

18. The method of claim 14, wherein the body length is combined length of both legs of the individual.

19. The method of claim 14, wherein the body length is length of one leg of the individual multiplied by 2.

20. The method of claim 14, wherein the walk comprises a speed of about 4.5 miles/hour or less.
